(19) United States
(12) Patent Application Publication
(10) Pub. No.: US 2025/0049423 A1
(43) Pub. Date: Feb. 13, 2025

(54) BIOPSY TOOL

(71) Applicant: INTUITIVE SURGICAL OPERATIONS, INC., Sunnyvale, CA (US)

(72) Inventors: Alain Sadaka, San Jose, CA (US); Shalv Madhani, Milpitas, CA (US); Hans Valencia, San Jose, CA (US)

(21) Appl. No.: 18/709,392

(22) PCT Filed: Nov. 10, 2022
(86) PCT No.: PCT/US2022/079658

§ 371 Date: May 10, 2024

Related U.S. Application Data
(60) Provisional application No. 63/278,677, filed on Nov. 12, 2021.

(51) Int. Cl. A61B 10/02

(52) U.S. Cl. CPC A61B 10/0275

(57) ABSTRACT

A biopsy tool includes a flexible cannula having a cutting surface on a distal end portion of the flexible cannula. In some examples, an atraumatic tip on a distal end portion of the flexible stylet extends from the distal end portion of the flexible cannula and at least partially shields the cutting surface of the flexible cannula when the biopsy tool is in a closed configuration.

### CROSS-REFERENCED APPLICATIONS

This application claims priority to and benefit of U.S. Provisional Application No. 63/278,677, filed Nov. 12, 2021 and entitled “Biopsy Tool,” which is incorporated by reference herein in its entirety.

### FIELD

The present disclosure is directed to systems and methods related to a biopsy tool.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions clinicians may insert minimally invasive medical instruments (including surgical, diagnostic, and/or therapeutic instruments) to reach a target tissue location. One such minimally invasive technique is to use a flexible and/or steerable elongate device, such as a flexible catheter that can be inserted into anatomic passageways and navigated toward a region of interest within the patient anatomy. Medical tools, such as biopsy instruments, may be deployed through the catheter to perform a medical procedure at the region of interest.

### SUMMARY

In one embodiment, a biopsy tool may include a flexible cannula having a cutting surface on a distal end portion of the flexible cannula; and a flexible stylet disposed at least partially within and extending from the distal end portion of the flexible cannula. The flexible stylet may have an atraumatic tip disposed on a distal end portion of the flexible stylet, where the atraumatic tip is configured to at least partially shield the cutting surface of the flexible cannula when the flexible cannula and the flexible stylet are in a closed configuration.

In one embodiment, a method for performing a biopsy may include: passing a biopsy tool through an internal channel of a medical device to a target biopsy site in a patient, wherein the biopsy tool comprises a flexible cannula having a cutting surface on a distal end portion of the flexible cannula and a flexible stylet disposed at least partially within and extending from the distal end portion of the flexible cannula; and at least partially shielding the cutting surface of the flexible cannula with an atraumatic tip of the flexible stylet as the biopsy tool is passed through the internal channel of the medical device.

In one embodiment, a biopsy tool may include a flexible cannula having a cutting surface on a distal end portion of the flexible cannula; and a flexible stylet disposed at least partially within and extending from the distal end portion of the flexible cannula. The flexible stylet may have an atraumatic tip at a distal end portion that extends proximally towards the cutting surface of the flexible cannula, where an outer maximum transverse dimension of the atraumatic tip is larger than an outer maximum transverse dimension of the flexible cannula at least at a leading distal end of the flexible cannula.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting examples when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 depicts one example of a biopsy tool;

FIGS. 2A-2D depict one example of a distal portion of a biopsy tool in a closed configuration;

FIGS. 2E-2F depict the distal portion of a biopsy tool of FIG. 2A in an open configuration;

FIG. 3 depicts a cross sectional view of one example of a distal portion of a biopsy tool in a closed configuration;

FIG. 4A depicts one example of a stylet with an atraumatic tip;

FIG. 4B depicts one example of a stylet with an atraumatic tip;

FIG. 5A depicts one example of a stylet with a sharp tip;

FIG. 5B depicts one example of a stylet with a sharp tip;

FIGS. 6A-6B depict one example of a distal portion of a biopsy tool with a sharp tip in an open configuration;

FIG. 7 depicts one example of an interior of a handle of a biopsy tool;

FIG. 8 depicts one example of a method for operating a biopsy tool;

FIG. 9 is a simplified diagram of an example of a robotic-assisted medical system; and

FIG. 10 is a simplified diagram of an example of a medical instrument system.

### DETAILED DESCRIPTION

A biopsy tool may be used to collect biopsy samples from a patient to diagnose a benign or malignant lesion. Larger biopsy samples with more layers of intact tissue may be required to definitively diagnosis a benign lesion than to diagnose a malignant lesion. If a large enough biopsy tissue sample is not collected, the patient may not receive a clear diagnosis of a suspicious lesion and may be subject to repeated and/or more invasive procedures. Conventional percutaneous devices for collecting lung biopsy samples from a patient typically include a rigid biopsy needle. A doctor guides the rigid biopsy needle from outside the patient's body, puncturing the patient's skin, underlying tissue, and lungs to position the rigid biopsy needle at the lesion site to collect a sample.

A flexible robotic-assisted medical system, or other flexible medical device, may permit access to the lungs or other organs via different routes (e.g., endoluminally through a patient's mouth, trachea, airway, etc.). However, conventional rigid biopsy tools may be incompatible with these medical systems and procedures. For example, lung biopsy procedures include inserting an elongated, flexible device with an internal channel (e.g., catheter, endoscope, laparoscope) into a patient's mouth through the airway to a target tissue site (e.g., lesion) in the lungs. As such, a flexible biopsy tool is desirable to permit the biopsy tool to be inserted through a narrow and sometimes tortuous internal channel of the elongated device to reach a distal opening of the elongated device at the target tissue.

Furthermore, conventional biopsy tools include a sharp distal tip to collect a tissue sample at the target site. In some instances, a sharp tip may damage the internal channel of the flexible device as the biopsy tool passes through the flexible device. More than one biopsy sample may be taken, so one or more biopsy tools may be inserted into and removed from the flexible device multiple times during a procedure. To prevent damage to the internal channel from the sharp tip as the biopsy tools pass through the channel in both directions, an outer protective sheath positioned between the biopsy tool and the surface of the internal channel is typically used to shield the internal channel from the sharp tip. However, the protective sheath around the biopsy takes up space in the internal channel and reduces the size of a biopsy tool that may fit through the narrow channel, resulting in smaller tissue sample sizes that may be collected by the narrower biopsy tool.

In view of the above, described herein are designs for a flexible biopsy tool that is compatible with elongated flexible devices having an internal channel. In some examples, the flexible biopsy tool includes a flexible cannula having a cutting surface on a distal end. A flexible stylet disposed at least partially within the cannula extends from a distal end portion of the flexible cannula. The stylet may include an atraumatic tip at a distal end of the stylet that shields the cutting surface of the cannula when the cannula and the stylet are in a closed configuration. As such, when in the closed position, the flexible biopsy tool may traverse through an elongated device to a distal opening of the device without risk of damage to the internal channel of the elongated device from the cutting surface. An outer protective sheath might not be used to protect the internal channel from the cutting surface of the biopsy tool in such an example, allowing the biopsy tool to be sized larger than biopsy tools that are used with outer sheaths.

In some examples, a flexible stylet of a biopsy tool includes a tissue-collecting notch that is exposed when the flexible cannula and the flexible stylet are in an open configuration. When a distal end of the biopsy tool reaches a distal opening of an associated elongated device and is positioned proximate to a tissue target site, the biopsy tool may be moved from the closed configuration to the open configuration (e.g., by moving the flexible cannula, the flexible stylet, or both). Once in the open configuration, the biopsy tool may be positioned to receive a portion of the target tissue into a volume of the notch. In the open configuration, the cutting surface of the cannula might no longer be shielded by the atraumatic tip, and the exposed cutting surface may be used to shear off a sample of the target tissue. For example, the biopsy device may be actuated back to the closed position (e.g., by firing the cannula, stylet, or both to move axially relative to each other), causing the cutting surface to shear off the target tissue positioned in the notch as the cutting surface and a distal end portion of the stylet are displaced toward each other.

In examples including the above noted atraumatic tip, the atraumatic tip may be sized and shaped to at least partially shield the cutting surface of the associated cannula to help prevent, or at least partially mitigate, the cutting surface catching on or cutting the internal channel of the elongated device as the biopsy tool travels through the device. In some examples, a proximal portion of the atraumatic tip may be shaped to conform to and match the shape of at least a portion of the cutting surface when the biopsy tool is in the closed configuration with the cutting surface positioned adjacent to the proximal portion of the atraumatic tip. In some examples, the atraumatic tip may have a larger diameter, or other maximum transverse dimension, than a corresponding maximum transverse dimension of a distal end portion of the cannula to prevent the cutting surface of the cannula from extending past the atraumatic tip. Thus, the cutting surface might only be exposed when the biopsy tool is moved to the open configuration. In some examples, a distal end portion of the atraumatic tip may be chamfered at an appropriate angle to allow the tip to safely navigate through an elongated internal channel and still allow the tip to puncture through tissue at a tissue target site.

In some examples, the disclosed flexible biopsy tool may be used to capture larger target tissue sample sizes as compared to conventional tools which may improve the accurate diagnoses of malignant and benign tumors. The disclosed flexible biopsy tools may also be compatible with a variety of flexible medical devices, including elongated devices with internal channels (e.g., catheter, endoscope, laparoscope, etc.). Regardless, in some examples, disclosed biopsy tools (e.g., without outer protective sheaths) may be navigated through narrow, tortuous elongated devices without risk of damage to the elongated devices in order to reach target tissue sites such as suspicious lesions throughout the body, including in the lungs and other organs. However, examples in which a sheath is used with a particular example of a biopsy tool are also contemplated as the disclosure is not so limited.

While some examples provided herein are related to usage of the disclosed biopsy tools with robotic-assisted surgical, diagnostic, and/or therapeutic procedures, any reference to medical or surgical instruments as well as medical or surgical methods is non-limiting. Specifically, the systems, instruments, and methods described herein may be used for manual operations, robotic-assisted operations, and/or any other desired usage. Additionally, the systems, instruments, and methods described herein may be used for operations related to humans, animals, human cadavers, animal cadavers, portions of human or animal anatomy, organ models, non-surgical diagnosis, as well as for industrial systems and general robotic, general teleoperational, robotic medical systems, and/or any other appropriate application as the disclosure is not limited in this manner.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

Turning to the figures, specific non-limiting examples are described in further detail. It should be understood that the various systems, components, features, and methods described relative to these examples may be used either individually and/or in any desired combination as the disclosure is not limited to only the specific examples described herein.

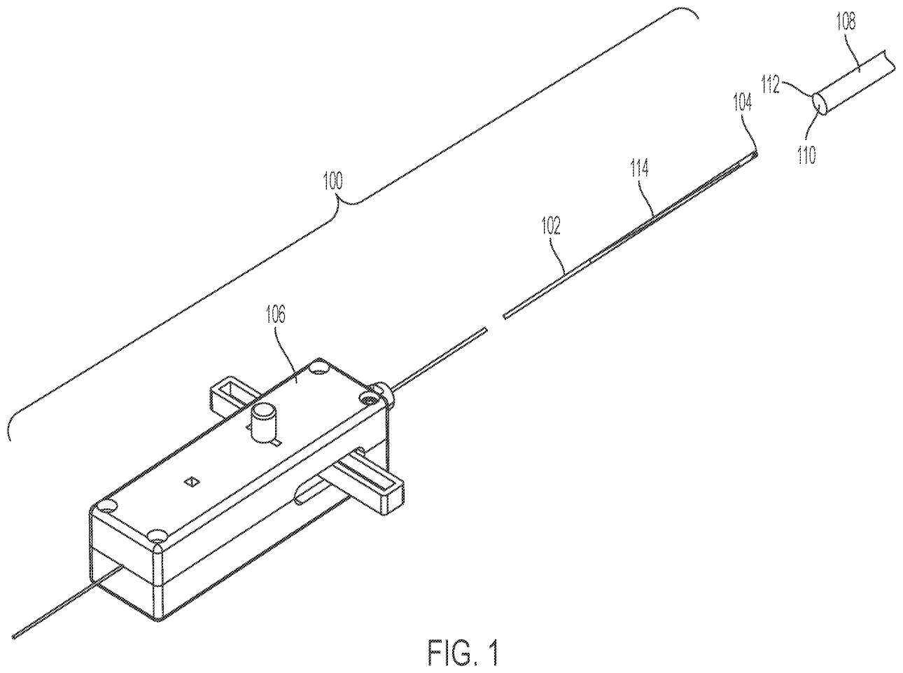

FIG. 1 shows a biopsy tool 100 according to one example. The biopsy tool 100 includes a flexible cannula 102 and a flexible stylet 104 at least partially received within a distal end portion of the flexible cannula 102. The biopsy tool 100 includes a handle portion 106 that the cannula and flexible stylet extend distally from. The handle portion may be used to help guide the cannula and stylet through an elongated device 108 and to control movement of the cannula and/or stylet to transition the biopsy tool between open and closed configurations, as described in further detail below. The handle portion 106 may be manually controlled by a physician or may be at least partially controlled using a robotic-assisted system.

As shown in FIG. 1, a distal end portion of the biopsy tool 100 may be inserted into a proximal opening 112 of the flexible elongated device 108 (e.g., catheter, endoscope, laparoscope, etc.) that has an internal channel 110 extending from the proximal opening to a distal opening (not shown). In some examples, the distal opening of the elongated device 108 may have been inserted into a patient, and the elongated device may have been navigated through the patient to a target tissue site. For example, the elongated device 108 may be a flexible catheter and the distal opening of the catheter may have been inserted into a patient's airway and the catheter guided through the lungs to reach a suspicious lesion in the lungs. The flexible catheter may have a small diameter to allow the catheter to travel a tortuous path to reach the suspicious lesion, which may be located deep within the lungs. In some examples, the elongated device may include an integrated imaging device or a separate imaging device to help navigate the catheter to its target location. If a separate imaging device is used, the imaging device may be removed from the internal channel 110 of the elongated device after the elongated device is in an appropriate position and prior to insertion of the biopsy tool 100 in some examples.

It should be noted that the disclosure is not limited to examples where a catheter is inserted into a patient's airway to access target tissue in the lungs, and any elongated device may be inserted into a patient (via a natural opening or through a patient's skin) to access any organ or tissue site in the patient. Reference to a catheter throughout the disclosure may also refer to other elongated devices with internal channels, such as an endoscope, laparoscope, and/or any other appropriate device including a channel through which the biopsy needle may be passed as the disclosure is not so limiting.

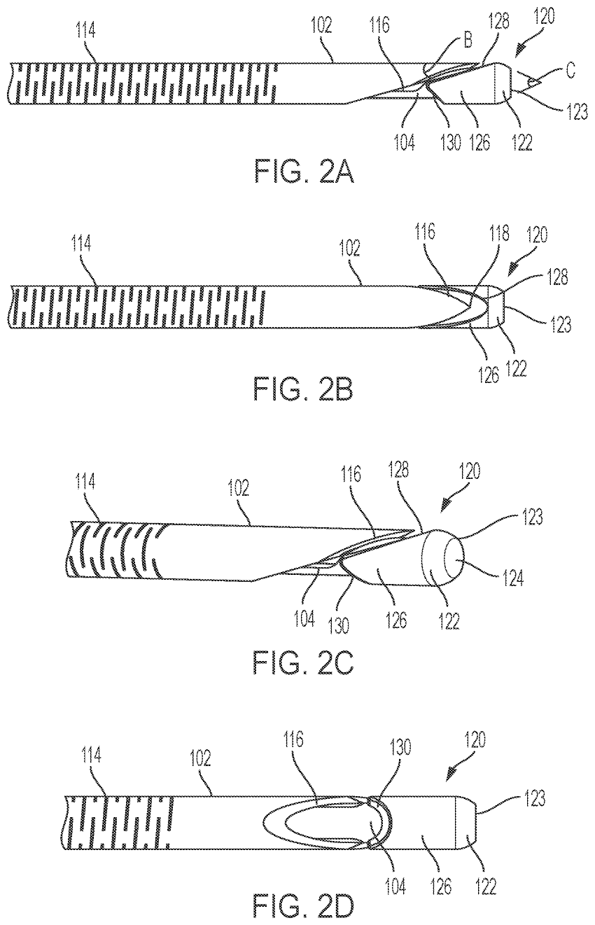

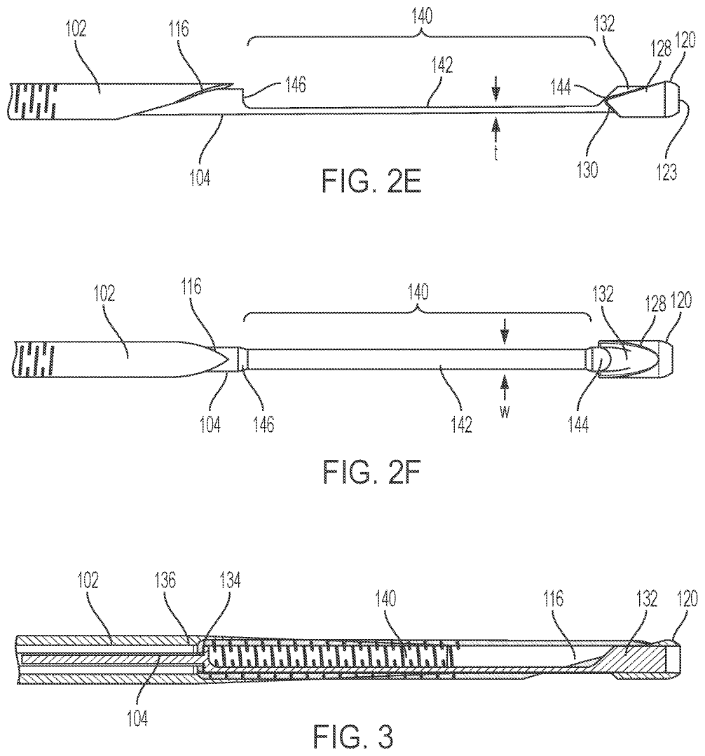

The cannula 102 may be flexible to follow the narrow (and sometimes tortuous) internal channel 110 of a catheter 108 that has been inserted into a patient and navigated to a target tissue site. Accordingly, as shown in FIGS. 1 and 2A-2B, the flexible cannula 102 may include a plurality of slits 114 to increase the flexibility of the cannula 102. The plurality of slits 114 may form living hinges to provide flexibility to allow the cannula to navigate the internal channel 110 of a catheter 108 to the target tissue, which may be located deep within a patient's lungs. In some examples, the plurality of slits 114 may extend to a proximal end of a cutting surface 116 of the cannula. However, in other examples, the plurality of slits may end prior to a proximal end of the cutting surface 116. Examples of biopsy tools including slits are described in U.S. Patent Application No. 2020/0077991, which is incorporated by reference herein in its entirety. Additionally, while a cannula including slits has been illustrated in the figure, other appropriate flexible constructions of the cannula are also contemplated including, for example, the use of flexible materials proximal to the cutting surface.

FIGS. 2A-2D are enlarged views of the distal portion of the biopsy tool of FIG. 1 with the cannula 102 and the stylet 104 in a closed configuration, according to some examples. A distal of end of the cannula 102 includes a cutting surface 116 for shearing off a portion of a target tissue when the cannula and stylet are moved from an open configuration to a closed configuration, as described in further detail below. As shown in FIG. 2A, the cutting surface may be angled relative to a longitudinal axis of the cannula to provide a force directed radially inwards to facilitate shearing off the target tissue. An angle B of the cutting surface 116 at the distal end of the cannula 102 (FIG. 2A) may be greater than or equal to approximately 10 degrees and/or less than or equal to approximately 45 degrees, though other angle ranges are also contemplated. Specifically, a smaller angle B may more easily shear off a portion of a target tissue, while a larger angle B may be easier to manufacture. As such, the angle B may be an appropriate angle to balance the case of shearing tissue with the case of manufacturing the cannula. In some examples, the angle B of the cutting surface 116 may be approximately 20 degrees. As shown in FIG. 2B, the angled cutting surface 116 provides a sharp distal tip 118 of the cannula that assists in piercing and cutting the target tissue.

As shown in FIGS. 2A-2D, a distal end portion of a stylet 104 extends from a distal end of the cannula 102, according to some examples. The distal end portion of the stylet 104 may include an atraumatic tip 120 that at least partially shields the cutting surface 116 of the cannula 102 when the cannula and stylet are in a closed configuration. Due to the configuration of the atraumatic tip 120, the biopsy tool 100 may be inserted into and traversed through a catheter, or other device including an internal channel, without risk of the sharp cutting surface 116 damaging (e.g., puncturing, tearing) the internal channel of the catheter. The biopsy tool 100 may therefore be used without an outer sheath positioned around the cannula to shield the cutting surface 116. This allows the cannula 102 to have a larger outer diameter than cannulas used with protective outer sheaths, which may allow the biopsy tool 100 to collect larger, intact tissue samples from the target tissue site.

In some examples, the outer diameter of the cannula 102 may be matched with the inner diameter of the internal channel of a catheter, while allowing for sufficient clearance for the cannula to traverse through the catheter while preventing undesirable movement of the cannula (e.g., bouncing around within the internal channel). In some examples, the outer diameter, or other appropriate maximum transverse dimension, of the cannula 102 may be between or equal to approximately 23-15 gauge (i.e., 0.65 mm and 1.8 mm), 17-15 gauge (i.e., about 1.4 mm and 1.8 mm), and/or any other appropriate size range. Such sizes may allow the biopsy tool 100 to collect much larger tissue sample sizes than biopsy tools having protective outer sheaths, which may include cannulas in the range of 19-18 gauge (i.e., 1.07 mm and 1.27 mm). However, other size ranges both greater and less than those noted above are also contemplated as the disclosure is not limited in this fashion.

As shown in FIGS. 2A-2D, in some examples the distal end portion 122 of the atraumatic tip 120 may be chamfered, slightly angled, and/or otherwise sized and shaped to allow the atraumatic tip 120 to puncture tissue while avoiding damaging an internal channel of a device when passed there through. In a nonlimiting example, suspicious lesions in the lungs (or other organs) may be extraluminal (e.g., outside an airway wall) and may require puncturing through tissue when a biopsy tool is deployed from an interior portion of the organ (e.g., via a lung airway) to reach the lesion. Therefore, the distal end portion 122 may be shaped to allow the atraumatic tip 120 to puncture through tissue to reach extra-luminal target tissue. Alternatively or additionally, a separate tool may be deployed through the internal channel of the elongate device to create a pilot hole in the target tissue site. The tool used to create the pilot hole may be removed prior to deploying the biopsy tool 100.

In some examples, the distal end portion 122 may be angled such that the distal end portion 122 is sharp enough to puncture tissue at a tissue target site, but not too sharp to damage the internal channel of a catheter as the biopsy tool 100 passes through the catheter. In some examples, the angle C of the distal end portion (see FIG. 2A) may be greater than or equal to approximately 30 degrees and/or less than or equal to approximately 40 degrees, or less than or equal to approximately 45 degrees. In some examples, the maximum outer diameter, or other maximum transverse dimension, of the distal end portion 122 of the atraumatic tip may be greater than or equal to about 1 mm and less than or equal to approximately 2 mm. However, the diameter, or other maximum transverse dimension, of the distal end 123 of the atraumatic tip may be greater than or equal to approximately 0.25 mm and/or less than or equal to approximately 0.5 mm. In some examples, the distal end portion 122 of the atraumatic tip 120 may have a length greater than or equal to about 0.5 mm and less than or equal to about 1 mm. While specific dimension ranges are provided above, any appropriate combination of dimensions, including dimensions both greater than and less than those noted above, are contemplated for a desired application as the disclosure is not so limited.

In some examples, as shown in FIG. 2C, the distal end of the atraumatic tip 120 may have a concave surface and may include an indent 124 extending into the distal end of the stylet. The indent 124 may help position and stabilize the biopsy tool when pushed into a target tissue site prior to tissue collection. In some examples, the distal end of the atraumatic tip may not be indented, and rather may have a convex, tapered, or a flat surface, as the disclosure is not so limiting. In some examples, the atraumatic tip may be shaped depending on the target tissue and application. In some examples, the atraumatic tip may have a bullet nose at a distal end.

As shown in FIG. 2A, in some examples, at least a portion of the atraumatic tip 120 includes an outer diameter, or other maximum transverse dimension, that is equal to or larger than an outer diameter, or other maximum transverse dimension, of a distal end portion of the cannula 102 (e.g., a leading distal end of the cutting surface 116). Thus, at least a distal end 118 of the cutting surface 116, and in some examples the entire cutting surface, of the cannula is disposed at or radially inwards from a corresponding portion of the atraumatic tip 120 in the closed configuration. When the biopsy tool is in a closed configuration, the atraumatic tip 120 may shield the cutting surface 116 to prevent damage to the internal channel of a catheter as well as unwanted cutting of unintended tissue. Alternatively or additionally, the biopsy tool 100 may include a mechanical stop to prevent the cutting surface 116 of the cannula from extending past the atraumatic tip 120, as described further below for FIG. 3. Examples in which a cannula includes a maximum transverse dimension that is greater than an associated maximum transverse dimension of the atraumatic tip are also contemplated. However, in such an example, a sheath may be used to shield the cutting surface from the surface of an internal channel of a device the biopsy tool is inserted into.

In some examples, and as shown in FIGS. 2A-2C, a proximal end portion 126 of the atraumatic tip 120 may have a shape that complements at least a portion of the cutting surface 116 when the cannula and the stylet are in a closed configuration. As shown in FIGS. 2A and 2C, a first surface 128 of the proximal end portion 126 is orientated in a first direction at an angle that complements an angle B of the cutting surface 116 with the first surface 128 forming a mating surface to a portion of the cutting surface 116. The first surface 128 may extend radially outwards from the stylet 104 such that when the cutting surface 116 of the cannula is mated with the first surface 128 of the atraumatic tip, the first surface 128 may extend radially up to or beyond an adjacent portion of the cutting surface 116 when in the closed configuration. Thus, in the closed configuration, the atraumatic tip 120 shields at least a portion of the cutting surface 116, including at least the sharp distal tip 118 at the distal end of the cannula. When the biopsy tool 100 is moved in a distal direction, either through a catheter or into target tissue, the atraumatic tip 120 shields the cutting surface 116 and the sharp distal tip 118 to prevent unwanted damage to the catheter or cutting of tissue.

As shown in FIGS. 2A-2D, the proximal end portion of the atraumatic tip 120 may also include a second surface 130 orientated in a second direction in some examples. For example, in the depicted example, the first surface 128 and the second surface 130 may form a rounded portion at the proximal end of the atraumatic tip 120. The rounded portion may allow the atraumatic tip 120 to be moved in a proximal direction through a catheter or tissue without catching or tearing the catheter or tissue while also permitting the atraumatic tip 120 to be easily manufactured. In some examples, the first surface 128 and the second surface 130 may be chamfered to further help prevent, or at least partially mitigate, the atraumatic tip 120 from catching tissue or a catheter when moved in a proximal direction.

As discussed above, in some examples, a biopsy tool 100 includes an atraumatic tip 120 that shields a cutting surface 116 on the distal end of the cannula 102 when the biopsy tool is in a closed configuration. The atraumatic tip 120 prevents damage to an internal channel of a catheter and unwanted cutting of tissue. Once the biopsy tool 100 has navigated through the catheter to a distal opening of the catheter and the atraumatic tip 120 has been positioned adjacent a target tissue site, the biopsy tool 100 may be moved to an open configuration to expose a notch 140 formed in the stylet 104 (e.g., as shown in FIG. 2E). The biopsy tool 100 may be moved from the closed configuration to the open configuration by axially moving the cannula 102, the stylet 104, or both relative to one another. For example, in some examples, the stylet 104 may move distally relative to the cannula 102, the cannula 102 may move proximally relative to the stylet 104, or both the cannula and stylet may be moved in these opposing directions towards the open configuration.

FIGS. 2E-2F show the cannula 102 and stylet 104 in an open configuration exposing a notch 140 that extends inwards into the stylet 104. In the open configuration, the distal end portion of the biopsy tool 100 may be positioned proximate a target tissue site to receive at least a portion of the target tissue into a volume of the notch 140. Once appropriately positioned, the biopsy tool 100 may be moved from the open configuration to the closed configuration, either by axially moving the cannula distally, the stylet proximally, or both. As the biopsy tool moves to the closed configuration, the cutting surface 116 of the cannula 102 may shear off the portion of the target tissue in the notch 140. The sheared off tissue may thereafter be stored within the notch 140, which is enclosed by the cannula 102 in the closed configuration.

As shown in FIGS. 2E-2F, notch 140 includes a spine 142 that extends between a distal side 144 and a proximal side 146 of the notch 140. The spine 142 may be flexible in order to conform to a shape of the flexible cannula 102 as the distal end portion of the biopsy tool traverses a narrow tortuous internal channel of a catheter or other device. However, the spine may be sufficiently rigid to facilitate the shearing off of tissue during a closing process as well as, in some examples, puncturing tissue to collect a tissue sample. Dimensions of the spine 142 may affect the rigidity of the spine 142 as well as the volume of the notch 140 to collect a target tissue sample. For example, increasing a thickness (t) and/or a width (w) of the spine 142 may increase the rigidity of the spine 142, but may also decrease the volume of the notch 140, limiting the size of the target tissue sample size collected by the biopsy tool 100. Similarly, increasing a length of the spine may increase a volume of the notch 140 while decreasing rigidity of the spine 142. Thus, the dimensions of the spine 142 and notch 140 may be selected to balance the desired rigidity of the spine while still collecting a large enough target sample size.

In some examples, a length of the spine 142 may be equal to or greater than approximately 5 mm and less than or equal to approximately 25 mm. In some examples, the length of the spine 142 may be between or equal to approximately 10 mm and 20 mm, 14 mm and 16 mm, or any other desired range. In one example, the spine may have a length of about 13 mm. In some examples, a thickness (t) of the spine may be equal to or greater than approximately 0.007 inches and less than or equal to approximately 0.015 inches. In some examples, a width of the spine 142 may be equal to or greater than approximately 0.025 and less than or equal to approximately 0.05 inches. Combinations of the forgoing dimensions, as well as dimensions both greater than and less than those noted above are contemplated as the disclosure is not so limited.

In some examples, the spine 142 may include a plurality of slits (not shown) along a length of the spine. The plurality of slits may increase the flexibility of the spine to allow for easier tracking of the stylet 104 with a shape of the flexible cannula 102 as the biopsy tool is passed through an internal channel of a catheter or other device. In some examples, a pattern for a plurality of slits on the spine may be similar to the pattern of the plurality of slits 114 in the cannula 102, shown in FIGS. 2A-2B. In other examples, the slits may correspond to a plurality of transverse or angled slits that extend partially across a width of the spine to form a plurality of living hinges along a length of the spine. Accordingly, it should be understood that any appropriate configuration of slits on the spine may be used. The plurality of slits may also allow excess blood or other liquids in a collected target tissue sample to drain through the slits away from the tissue sample after the target tissue is collected and stored in the biopsy tool 100. In some examples, the slits in the spine 142 may have a greater width than the plurality of slits 114 in the cannula 102 to help blood or other liquids drain away from a tissue sample. Examples in which a solid spine made from a sufficiently elastic material is used are also contemplated.

As shown in FIGS. 2E-2F, one or both of the distal side 144 and proximal side 146 of the notch 140 may be angled outwards from the notch 140 such than an outer opening to the notch 140 may have a larger axial length than a bottom surface of the notch 140 which may correspond to an inner surface of the spine. This increased opening size may assist in target tissue collection. The angled sides may allow tissue to enter the notch easily when the stylet is pressed into a target tissue site. Moreover, the sides 144 and 146 may hold and stabilize the target tissue in the notch 140 when the cannula 102 and stylet 104 are moved from an open configuration to a closed configuration to shear off the target tissue. In some examples, the outward angling of the distal side 144 may allow the cutting surface 116 of the cannula to slide smoothly over the side 144 without catching as the biopsy tool is moved to a closed position. In some examples, the distal side 144 and proximal side 146 may form an angle with the spine that is equal to or greater than approximately 90 degrees and less than or equal to approximately 135 degrees though other angular ranges may also be used.

In some examples, the biopsy tool may include, or be configured to be connected to, a vacuum source. During operation, the vacuum source may apply a suction to at least a portion of the interior volume of the notch to bias a portion of target tissue into the notch. The vacuum source may include a vacuum lock, an active pump, or any other appropriate source of suction. The suction may be provided through a channel in the cannula 102, a tube extending through the cannula to the notch, a channel formed in the stylet 104, or any other construction that provides fluid communication between a vacuum source and the notch to apply the desired suction to at least a portion of the notch.

As mentioned above, the stylet 104 may be rigid enough to allow the biopsy tool to puncture through tissue and to move through an internal channel of a catheter. In some examples, a stylet 104 may include a solid portion 132 that is received at least partially within a distal end portion of the cannula 102. In the depicted example, an atraumatic tip 120 may correspond to a collar press fit on, or otherwise attached to, the solid portion of the stylet to provide the desired shielding of the cutting surface of the cannula. In the closed configuration, as shown in FIG. 3, a transverse cross-section of the distal end portion of the cannula 102 may be sized and shaped to complement a transverse cross-section of the solid portion 132 received therein. The mating of these surfaces may improve a rigidity of the distal end of the biopsy tool 100 when the biopsy tool is in the closed configuration with the solid portion of the stylet received at least partially in the cannula. This increased rigidity may aid in puncturing tissue and maintaining a stable configuration between the cannula and stylet during insertion and removal from an interior channel of a catheter or other device.

As mentioned above, in some examples, the biopsy tool 100 may include a mechanical stop to prevent a cutting surface 116 of a cannula 102 from extending past an atraumatic tip 120 of a stylet 104. As shown in FIG. 3, a stylet 104 includes a stop 134, such as a ledge, protrusion, detent, or other appropriate feature positioned proximal to a notch 140 of the stylet. The stop engages with (e.g., abuts against) a corresponding surface 136 on an interior of the cannula 102 when the stylet and cannula are in a closed position which prevents further distal extension of the cannula relative to the stylet. In such examples, the atraumatic tip 120 may or may not have a larger outer diameter than a diameter of a distal end of the cannula. Additionally, depending on the desired application, sharp tips may be used in place of the atraumatic tip. In either case, if the tip 120 has a smaller diameter than the distal end portion of the cannula, the stop 134 may prevent the cannula from extending past the tip. It should be noted that the illustrated example is one of many variations of a mechanical stop that may be included to limit axial movement of the cannula relative to the stylet, as the disclosure is not so limiting. Additionally, a mechanical stop may be positioned along any portion of the biopsy tool between a proximal end (e.g., at a handle portion) and a distal end of the device as the disclosure is not limited to any particular construction or location of the mechanical stop.

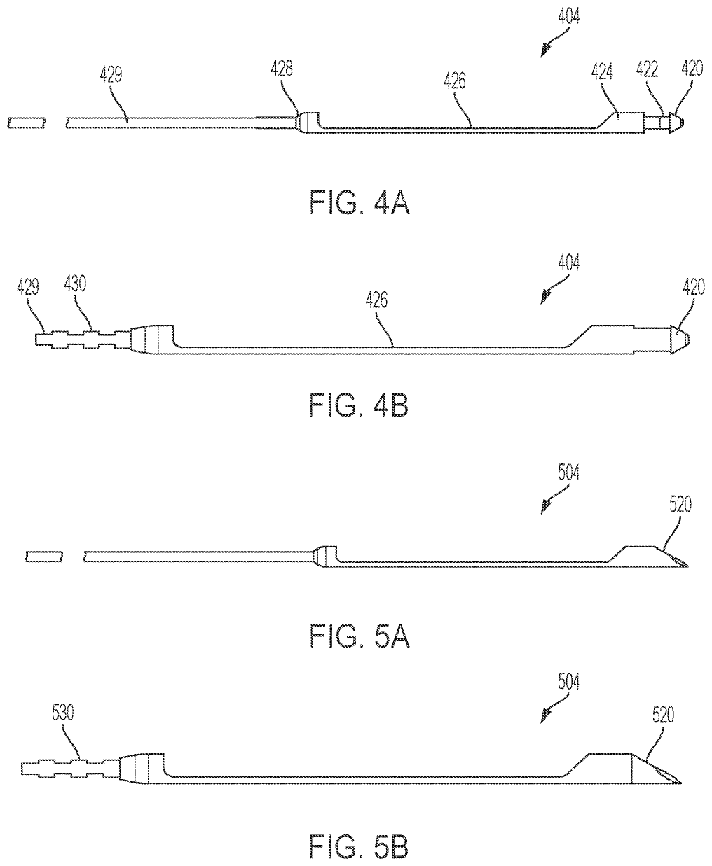

FIGS. 4A-4B, 5A-5B, and 6A-6B show different examples of a stylet that may be used in a biopsy tool 100. As shown in FIGS. 4A-4B, a stylet 404 may include an atraumatic tip 420 attached to a distal end of the stylet via a connector 422. The connector 422 may extend between a solid portion 424 of the stylet and the atraumatic tip 420. The solid portion 424 may provide a mating surface for an inner surface of a distal end of a cannula to help stabilize the stylet within the cannula in the closed configuration as discuss above. The connector 422 may have a smaller diameter than a maximum outer diameter of the atraumatic tip 420 and the solid portion 424. The stylet 404 may also include a mechanical stop, such as surface 428, configured to abut an inner surface of a cannula to prevent the cannula from extending past the atraumatic tip 420.

In some examples, the stylet 404 may include a proximal segment 429 positioned proximal to a notch 426 of the stylet. The proximal segment 429 may be smaller in diameter than the atraumatic tip to increase the flexibility of the stylet 404 and allow for better tracking in a cannula. As shown in FIG. 4B, in some examples, a joint between the proximal segment 429 and the notch 426 stylet 404 may include a ribbed portion 430 to increase the strength of the joint while maintaining flexibility. The ribbed portion 430 may be made of plastic and may be hollow or include a metal core for improved strength and pushability.

In some examples, as shown in FIGS. 5A-5B, a stylet 504 may include a sharp tip 520. The sharp tip 520 may include an angled surface, or other structure, that is configured to puncture through tissue similar to a biopsy needle tip. In such examples, an outer sheath (not shown) may be used to surround a cannula and sharp tip 520 while guiding a biopsy tool through an interior channel of a catheter or other device and into a target tissue site to reduce risk of damage to the catheter or tissue. The outer sheath may be removed from the biopsy tool once the biopsy tool is positioned proximate the target tissue site and prior to collecting a tissue sample. Similar to FIG. 4B, in some examples, stylet 504 may include a ribbed portion 530 positioned proximate to a notch 526 of the stylet 504.

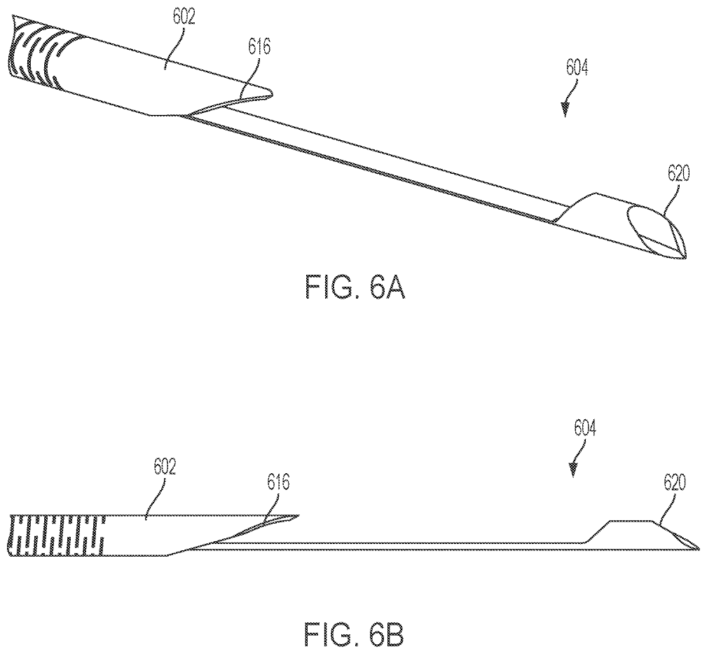

In some examples, as shown in FIGS. 6A-6B, a stylet 604 may include a tri-bevel tip 620 that is configured to pierce tissue. The tri-bevel tip 620 includes three surfaces that extend distally from a sharp point at the distal end of the stylet 604 to allow the tip 620 to puncture tissue. Similar to the stylet 504 of FIGS. 5A-5B, a biopsy tool using stylet 604 may be used with an outer sheath to shield the traumatic tip 620 to prevent damage to an internal channel of a catheter or tissue. In examples including a sharp tip such as that shown in the figures, the biopsy tool 100 may again include a mechanical stop to prevent a cutting surface 616 of a cannula 602 from extending distally past the traumatic tip 620 of the stylet 604 as previously discussed.

In the examples described above, a cannula and/or a stylet of a biopsy tool may be made using metal, plastic, a combination thereof, and/or any other appropriate material. For example, both the cannula and stylet may be made of metal (e.g., stainless steel, nitinol, any clastic biocompatible alloy, spiral cut stainless steel, coil pipe stainless steel, etc.). Alternatively, one of the cannula, stylet, or both may be made of a plastic (e.g., PEEK, nylon, polyethylene, etc.). However, it should be understood that the cannula and/or stylet may be made of any material that provides sufficient flexibility to traverse through a narrow, tortuous catheter, while also being rigid enough to collect a tissue sample, and in some examples, puncture through tissue.

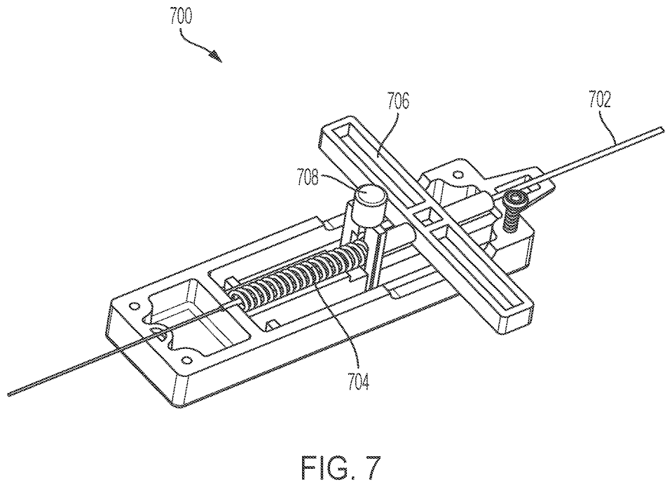

FIG. 7 shows an example of a handle 700 that may be used with a biopsy tool described herein. The handle 700 may be coupled with a proximal end portion of a cannula 702 that may extend distally out from the handle 700. The handle 700 may be controlled manually (e.g., by a physician in a room with a patient undergoing a biopsy procedure), robotically-assisted, or telcoperatively controlled (e.g., by a physician located inside or outside the room). The handle 700 may include a spring 704 operatively coupled to a stylet (not shown) disposed within and extending through the cannula. The spring may bias the cannula and stylet to a closed configuration as described above. It should be noted that the spring 704 is a non-limiting example, and the cannula and stylet may be biased to either a closed or open configuration using any other appropriate construction. As shown in FIG. 7, the handle 700 may include a draw handle 706 that is coupled to either the cannula or stylet such that the cannula or stylet is configured to axially move with the draw handle 706. For example, the draw handle 706 may be coupled to the cannula such that when the draw handle is moved in a proximal direction, the cannula moves axially in a proximal direction relative to the stylet to move the biopsy tool to an open configuration and expose a notch in the stylet. Moving the draw handle 706 in a proximal direction may compress the spring 704 to load the spring. The draw handle 706 may be locked in the proximal position to resist the force of the spring and to maintain the biopsy tool in the open configuration by a lock 708. The lock 708 is depicted as a button lock that, when depressed, may release the draw handle 706 from the locked proximal position, allowing spring 704 to push the draw handle 706 back to its distal position. As the draw handle is moved distally by the spring, the cannula is correspondingly moved in the distal direction back to the closed configuration. The force of the spring may cause the cannula to move with a sufficient force to shear off a portion of the target tissue positioned in the notch. While a particular actuation mechanism is depicted in FIG. 7, it should be noted that the disclosure is not so limiting, and other actuation or firing mechanisms may be used to move the biopsy tool between the disclosed open and closed configurations.

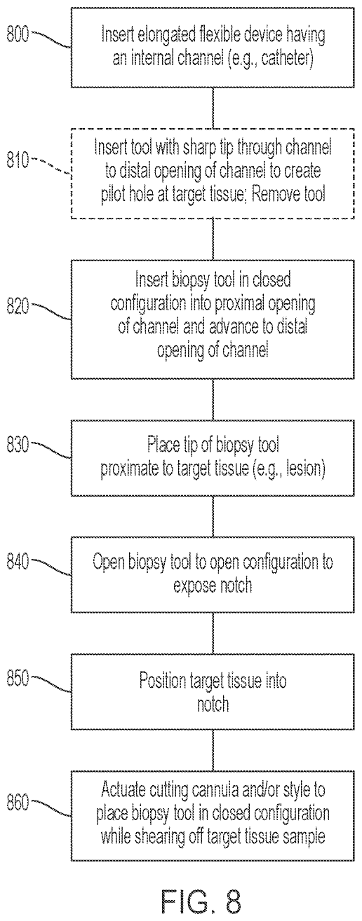

FIG. 8 is a method of using a biopsy tool according to some examples. In step 800, an elongated flexible device having an internal channel is inserted into a patient. The elongated device may be a catheter, endoscope, laparoscope, or other appropriate device with an internal channel. The elongated device may include a proximal opening and a distal opening. The portion of the device including the distal opening is inserted into a patient and navigated to a target tissue site in the patient. In some examples, the device may be inserted into a patient's airway and navigated to a target tissue site in the patient's lungs. However, it should be noted that the elongated device may be inserted into any natural orifice of a patient or through an incision in the patient, and navigated to any organ or tissue target site, as the disclosure is not so limiting. In some examples, the elongated device may include an integrated imaging device to provide a visual aid when navigating the device to a target tissue site. Alternatively, a separate imaging device may be inserted into and used with the elongated device to provide visual assistance during initial positioning of the elongated device. If a separate imaging device is used, the probe may be removed once the elongated device is in a position and orientation relative to a target tissue site.

In optional step 810, a tool having a sharp distal tip may be inserted through the internal channel of the elongated device to the distal opening. The tool may be used to create a pilot hole at the tissue target site. A pilot hole may be useful to assist a biopsy tool having an atraumatic tip in puncturing through tissue at the target site. After creating a pilot hole, the tool is then removed. The tool may be inserted prior to inserting the elongated device into the patient or after the elongated device is inserted. An outer protective sheath may be used with the sharp tool to prevent the sharp tool from damaging the internal channel of the elongated device.

In step 820, a biopsy tool is inserted into the proximal opening of the elongated device and advanced through the internal channel to the distal opening. The biopsy tool may be biased towards a closed configuration while traversing the internal channel. In the closed configuration, an atraumatic tip on the distal end portion of a stylet may shield a cutting surface on a distal end of a cannula to protect the internal channel from damage. However, examples in which a sharp distal tip is used are also contemplated.

In step 830, the atraumatic tip at the distal end of the biopsy tool is positioned proximate to the target tissue site. The tissue site may be a suspicious lesion in a patient, such as in the lungs or other organ. Positioning the atraumatic tip proximate to target tissue may include puncturing through tissue to reach a suspicious lesion (e.g., an extraluminal lesion in the lungs). In step 840, the biopsy tool is moved to an open configuration to expose a notch in the stylet. The biopsy tool may be moved to the open configuration by axially moving the cannula in a proximal direction, or axially moving the stylet in a distal direction, or a combination of both. Alternatively, the tip of the biopsy tool may be pushed into a target tissue (step 830) while still in the closed configuration, and then moved to the open configuration (step 840).

Once in the open configuration, in step 850, the biopsy tool is positioned to receive a portion of the target tissue into the exposed notch. In some examples, the catheter, or other device, may be slightly articulated in a direction of the notch (e.g., manually or via software if robotically assisted) to precisely position the target tissue into the notch with increased control. In some examples, the biopsy device may include or be connected to a vacuum source through a channel in the cannula and/or stylet to apply a suction at the notch to ensure a portion of the target tissue is well seated in the notch.

In step 860, the biopsy tool is actuated to move the cannula and stylet to the closed configuration. In some examples, the cannula is actuated to move axially in a distal direction with sufficient force, causing the cutting surface on the distal end of the cannula to shear off the target tissue positioned in the notch. Alternatively, the stylet may be actuated to move axially in a proximal direction, or both the cannula and stylet may be actuated simultaneously. In some examples, a handle of the biopsy tool may include an actuator to actuate the biopsy tool between the open and closed configurations. The biopsy tool may include any actuation system including, for example, a servo, inputs from a separate robotic system, a separate power pack, the spring and draw handle arrangement described above, and/or any other appropriate actuator as the disclosure is not so limiting.

The biopsy device may store the sheared off target tissue in the notch, protected by the inner surface of the cannula when in the closed configuration. The biopsy tool may then be removed from the catheter to retrieve the target sample. The same or different biopsy tool may be inserted to retrieve multiple target tissue samples.

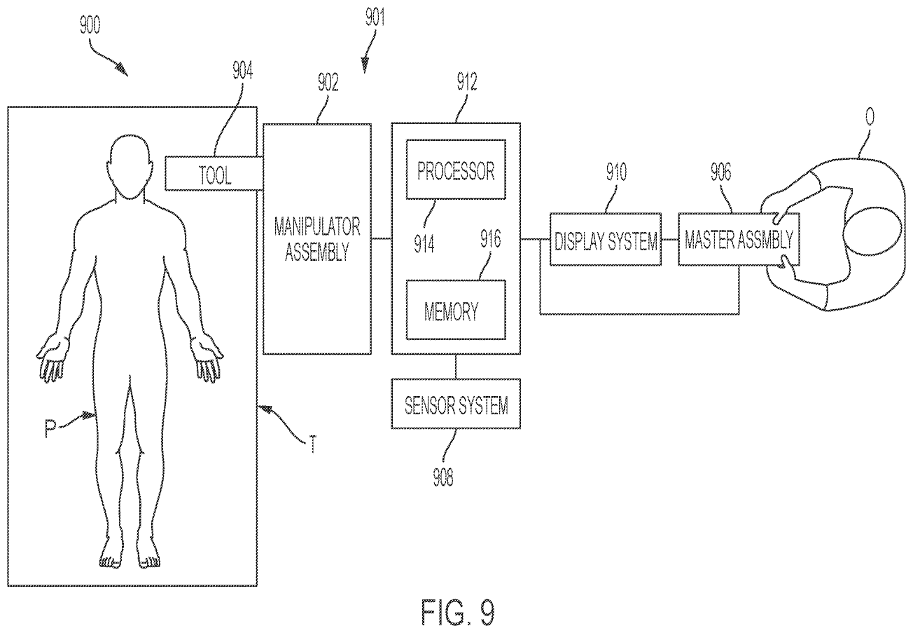

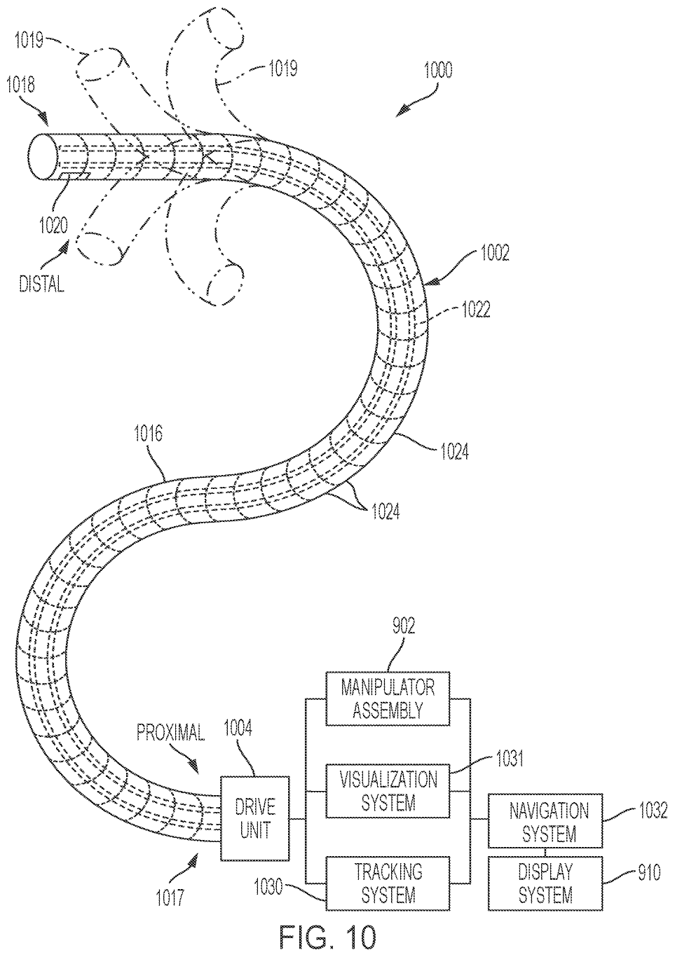

In some examples, the medical tools disclosed herein may be used in a medical procedure performed with a robotic-assisted medical system as described in further detail below. As shown in FIG. 9, a robotic-assisted medical system 900 may include a manipulator assembly 902 for operating a medical tool 904 in performing various procedures on a patient P positioned on a table T in a surgical environment 901. The medical tool 904 may correspond to any one of the medical tools described herein (e.g., medical tool 100) or any other medical tool within the scope of this description. Additionally or alternatively, the medical tool 904 may be a catheter having a lumen, as will be described in further detail with reference to FIG. 10. In these examples, the medical tool may be inserted into the lumen of the medical tool 904. The manipulator assembly 902 may be robotic-assisted, manually operated, or a hybrid assembly with select degrees of freedom of motion that may be motorized and/or select degrees of freedom of motion that may be non-motorized. A master assembly 906, which may be inside or outside of the surgical environment 901, generally may include one or more control devices for controlling manipulator assembly 902. Manipulator assembly 902 supports medical tool 904 and may include a plurality of actuators or motors that drive inputs on medical tool 904 in response to commands from a control system 912. The actuators may include drive systems that when coupled to medical tool 904 may advance medical tool 904 into a naturally or surgically created anatomic orifice. Other drive systems may move the distal end of medical tool in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical tool 904 for grasping tissue in the jaws of a biopsy device and/or the like.

Robotic-assisted medical system 900 also may include a display system 910 for displaying an image or representation of the surgical site and medical tool 904 generated by a sensor system 908 which may include an endoscopic imaging system. Display system 910 and master assembly 906 may be oriented so an operator O can control medical tool 904 and master assembly 906 with the perception of telepresence. Any of the previously described graphical user interfaces may be displayable on a display system 910 and/or a display system of an independent planning workstation.

In some examples, medical tool 904 may include components for use in surgery, biopsy, ablation, illumination, irrigation, or suction. Optionally medical tool 904, together with sensor system 908 may be used to gather (e.g., measure or survey) a set of data points corresponding to locations within anatomic passageways of a patient, such as patient P. In some examples, medical tool 904 may include components of the imaging system which may include an imaging scope assembly or imaging instrument that records a concurrent or real-time image of a surgical site and provides the image to the operator or operator O through the display system 910. In some examples, imaging system components may be integrally or removably coupled to medical tool 904. However, in some examples, a separate endoscope, attached to a separate manipulator assembly may be used with medical tool 904 to image the surgical site. The imaging system may be implemented as hardware, firmware, software or a combination thereof which interact with or are otherwise executed by one or more computer processors, which may include the processors of the control system 912.

The sensor system 908 may include a position/location sensor system (e.g., an electromagnetic (EM) sensor system) and/or a shape sensor system for determining the position, orientation, speed, velocity, pose, and/or shape of the medical tool 904.

Robotic-assisted medical system 900 may also include control system 912. Control system 912 may include at least one memory 916 and at least one computer processor 914 for effecting control between medical tool 904, master assembly 906, sensor system 908, and display system 910. Control system 912 also may include programmed instructions (e.g., a non-transitory machine-readable medium storing the instructions) to implement a plurality of operating modes of the robotic-assisted medical system including a navigation planning mode, a navigation mode, and/or a procedure mode. Control system 912 also may include programmed instructions (e.g., a non-transitory machine-readable medium storing the instructions) to implement some or all of the methods described in accordance with aspects disclosed herein, including, for example, moving a mounting bracket coupled to the manipulator assembly to the connection member, processing sensor information about the mounting bracket and/or connection member, and providing adjustment signals or instructions for adjusting the mounting bracket.

Control system 912 may further include a virtual visualization system to provide navigation assistance to operator O when controlling medical tool 904 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

FIG. 10 is a simplified diagram of a medical instrument system 1000 according to some examples. Medical instrument system 1000 may include elongate device 1002, which may be the same as or similar to medical tool 904 of FIG. 9 or any sheath described herein, coupled to a drive unit 1004. Elongate device 1002 may include a flexible body 1016 having proximal end 1017 and distal end or tip portion 1018. Medical instrument system 1000 further may include a tracking system 1030 for determining the position, orientation, speed, velocity, pose, and/or shape of distal end 1018 and/or of one or more segments 1024 along flexible body 1016 using one or more sensors and/or imaging devices as described in further detail below.

Tracking system 1030 may optionally track distal end 1018 and/or one or more of the segments 1024 using a shape sensor 1022. Shape sensor 1022 may optionally include an optical fiber aligned with flexible body 1016 (e.g., provided within an interior channel (not shown) or mounted externally). The optical fiber of shape sensor 1022 forms a fiber optic bend sensor for determining the shape of flexible body 1016. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some examples may employ other suitable strain sensing techniques, such as Rayleigh scattering, Raman scattering, Brillouin scattering, and Fluorescence scattering. In some examples, the shape of the elongate device may be determined using other techniques. For example, a history of the distal end pose of flexible body 1016 can be used to reconstruct the shape of flexible body 1016 over the interval of time. In some examples, tracking system 1030 may optionally and/or additionally track distal end 1018 using a position sensor system 1020. Position sensor system 1020 may be a component of an EM sensor system with position sensor system 1020 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 1020 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

Flexible body 1016 may include a channel sized and shaped to receive a medical instrument. In various examples, any of the instruments and sheaths described above may be inserted through the channel of the flexible body 1016. For example, any one of the medical tools described herein may be inserted into the channel of the flexible body 1016. Medical instruments may include, for example, image capture probes, biopsy instruments, laser ablation fibers, and/or other surgical, diagnostic, or therapeutic tools. Medical instruments may be used with an imaging instrument (e.g., an image capture probe) also within flexible body 1016.

Flexible body 1016 may also house cables, linkages, or other steering controls (not shown) that extend between drive unit 1004 and distal end 1018 to controllably bend distal end 1018 as shown, for example, by broken dashed line depictions 1019 of distal end 1018. In some examples, at least four cables are used to provide independent “up-down” steering to control a pitch of distal end 1018 and “left-right” steering to control a yaw of distal end 1018. Steerable elongate devices are described in detail in U.S. patent application Ser. No. 13/274,208 (filed Oct. 14, 2011) (disclosing “Catheter with Removable Vision Probe”), which is incorporated by reference herein in its entirety.

The information from tracking system 1030 may be sent to a navigation system 1032 where it is combined with information from image processing system 1031 and/or the preoperatively obtained models to provide the operator with real-time position information. In some examples, the real-time position information may be displayed on display system 910 of FIG. 9 for use in the control of medical instrument system 1000. In some examples, control system 912 of FIG. 9 may utilize the position information as feedback for positioning medical instrument system 1000. Various systems for using fiber optic sensors to register and display a surgical instrument with surgical images are provided in U.S. patent application Ser. No. 13/107,562, filed May 13, 2011, disclosing, “Medical System Providing Dynamic Registration of a Model of an Anatomic Structure for Image-Guided Surgery,” which is incorporated by reference herein in its entirety.

In some examples, medical instrument system 1000 may be robotic-assisted within medical system 900 of FIG. 9. In some examples, manipulator assembly 902 of FIG. 9 may be replaced by direct operator control. In some examples, the direct operator control may include various handles and operator interfaces for hand-held operation of the instrument.

While several examples of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be illustrative and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific examples of the disclosure described herein. It is, therefore, to be understood that the foregoing examples are presented by way of example only and that, within the scope of the appended claims and equivalents thereto, the disclosure may be practiced other than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A biopsy tool comprising:
a flexible cannula having a cutting surface on a distal end portion of the flexible cannula; and
a flexible stylet disposed at least partially within and extending from the distal end portion of the flexible cannula, the flexible stylet having an atraumatic tip disposed on a distal end portion of the flexible stylet, wherein the atraumatic tip is configured to at least partially shield the cutting surface of the flexible cannula when the flexible cannula and the flexible stylet are in a closed configuration.

2. The biopsy tool of claim 1, wherein the atraumatic tip is configured to puncture tissue of a patient.

3. The biopsy tool of claim 1, wherein a shape of a proximal end portion of the atraumatic tip is configured to complement a shape of at least a portion of the cutting surface when the flexible cannula and the flexible stylet are in the closed configuration.

4. The biopsy tool of claim 3, wherein the proximal end portion includes a first surface oriented in a first direction and a second surface oriented in a second direction, wherein the first direction and the second direction are oriented at least partially in a proximal direction.

5. The biopsy tool of claim 1, wherein the flexible stylet includes a notch configured to be exposed to an exterior environment when the flexible cannula and flexible stylet are in an open configuration and disposed within the flexible cannula when the flexible cannula and flexible stylet are in the closed configuration.

6. The biopsy tool of claim 5, wherein a distal portion of the notch is angled to prevent the cutting surface from catching the distal portion of the notch as the flexible cannula and the flexible stylet move from the open configuration to the closed configuration.

7. The biopsy tool of claim 5, wherein the flexible stylet includes a plurality of slits disposed along a spine of the notch.

8. The biopsy tool of claim 1, wherein an outer maximum transverse dimension of the atraumatic tip is larger than an outer maximum transverse dimension of the flexible cannula at least at a leading distal end of the flexible cannula.

9. The biopsy tool of claim 1, wherein a distal end portion of the atraumatic tip is chamfered.

10. The biopsy tool of claim 1, wherein the biopsy tool is configured to apply suction to tissue proximate to the flexible stylet when the flexible cannula and the flexible stylet are in an open configuration.

11. The biopsy tool of claim 1, wherein the flexible cannula includes a plurality of slits.

12. The biopsy tool of claim 11, wherein the plurality of slits of the flexible cannula extend to a proximal end of the cutting surface.

13. The biopsy tool of claim 11, wherein the plurality of slits of the flexible cannula end prior to a proximal end of the cutting surface.

14. The biopsy tool of claim 1, wherein the flexible cannula and the flexible stylet are biased to the closed configuration.

15. The biopsy tool of claim 14, wherein the flexible cannula and the flexible stylet are biased to the closed configuration by a spring loaded actuator.

16. The biopsy tool of claim 1, wherein an outer maximum transverse dimension of the atraumatic tip disposed on the distal end portion of the flexible stylet is between approximately 1 mm and 2 mm.

17. The biopsy tool of claim 1, wherein the biopsy tool does not include a sheath.

18.-43. (canceled)

44. The biopsy tool of claim 5, wherein the flexible stylet further includes a stop surface proximal of the notch, the stop surface configured to restrict distal motion of the flexible cannula relative to the flexible stylet.

45. The biopsy tool of claim 44, wherein the flexible cannula further includes a protruding surface proximal of the cutting surface, wherein the protruding surface is configured to abut the stop surface to restrict distal motion of the flexible cannula relative to the flexible stylet.

46. The biopsy tool of claim 1, flexible stylet includes a solid portion extending between the notch and the atraumatic tip, the solid portion configured to be at least partially received within the distal end portion of the flexible cannula.
